(19) United States
(12) Patent Application Publication
(10) Pub. No.: US 2020/0101470 A1
(43) Pub. Date: Apr. 2, 2020

(54) MAGNETIC BAR CAPTURE DEVICE

(71) Applicant: The United States of America, as Represented by the Secretary of Agriculture, Washington, DC (US)

(72) Inventors: CHERYL M. ARMSTRONG, WYNDMOOR, PA (US); JOSEPH A. CAPOBIANCO, Jr., MARLTON, NJ (US); ANDREW G. GEHRING, DRESHER, PA (US)

(21) Appl. No.: 16/582,750

(22) Filed: Sep. 25, 2019

Related U.S. Application Data
(60) Provisional application No. 62/737,212, filed on Sep. 27, 2018.

(51) Int. Cl. B03C 1/01; B03C 1/28; C12N 13/00

(52) U.S. Cl. CPC B03C 1/01; B03C 1/28; B03C 2201/26; B03C 2201/18; C12N 13/00

(57) ABSTRACT

A single macroscopic magnetic capture device may be functionalized and utilized to query a volume of liquid for a particular target analyte. The magnetic capture device may be rotated to create a vortex to enhance the efficiency of capture. The magnetic capture device may include a ferromagnetic element and a bioactive coating affixed to the surface of the ferromagnetic element, the bioactive coating being configured to capture the target analyte. In some embodiments, a capture container may be utilized together with the magnetic capture device, the geometries of the magnetic capture device and capture container being predetermined to effect a desired fluid dynamic system within the capture container. A customizable kit for allowing a user to create a custom magnetic capture device is also contemplated.

### REFERENCE TO RELATED APPLICATIONS

This application claims the benefit of U.S. Provisional Application No. 62/737,212, filed Sep. 27, 2018, which is incorporated herein by reference in its entirety.

### BACKGROUND

The idea behind using iron-containing microspheres to capture and subsequently isolate cells of interest from a mixed population was published in 1977 (Molday et al. Application of magnetic micro spheres). Since that time, these particles have become integrated into hundreds of applications due to their proven track record for isolating and concentrating cells, proteins, and/or nucleic acids of interest from assorted matrices.

Commercial magnetic particles are typically small (<10 μm in diameter). It is a technological challenge to control size, shape, stability, and dispersibility of these particles in desired solvents. Consequently, the final particles are irregularly shaped, polydisperse particles that tend to aggregate so as to minimize surface energies. Further, in the separation protocols for these particles, the particles are subjected to a magnetic force to hold them against other forces such as gravity and hydrodynamic forces. These small particles are generally weakly magnetic (i.e. antiferromagnetic or paramagnetic) and require separating devices using high gradient magnetic separation (also known as an HGM technique) for efficient separation.

Although numerous methods have been developed for preparing small magnetic beads, the chemical synthetic method developed by Ugelstad in 1970s remains the most successful route so far and has successfully been used for creating commercialized magnetic beads. The process, described in U.S. Pat. No. 4,654,267, has been demonstrated to produce spherical magnetic polymer microbeads with a narrow particle size distribution. However, the method is not without limitations. In particular, beads in this size regime are prone to quick sedimentation due to gravity, which is unfavorable for the separation efficiency.

Studies indicate that the capture efficiency of the beads is largely controlled by the rate of interaction of the bead with the target particle to be captured, which is related to the sedimentation volume of the beads themselves (Irwin et al. Immunomagnetic bead mass transport, 2002). Since the rate of interaction is lower on a particle-by-particle basis when an equal number of particles to be captured are placed in a large volume compared to a small volume, more beads must be used to query the larger volume if an equivalent number of particles are to be captured in the same amount of time. Cost can then quickly become prohibitive when using the beads in routine diagnostic testing requiring large sample volumes. Until cheaper alternatives are discovered, assay development for large volume samples will likely be hindered by cost.

All of the references cited herein, including U.S. Patents and U.S. Patent Application Publications, are incorporated by reference in their entirety.

Mention of trade names or commercial products in this publication is solely for the purpose of providing specific information and does not imply recommendation or endorsement by the U.S. Department of Agriculture.

### SUMMARY

In an effort to apply the basic principles behind immunomagnetic separation to large volume samples, the present invention uses a single macroscopic magnetic particle which may be rotated to create a vortex or agitated in some other way to enhance the efficiency of capture. The use of a single macroscopic particle makes assaying large volumes economical because it reduces the costs associated with particle production by incorporating an inexpensive manufacturing process into the fabrication of the capture device and reduces the overall cost associated with the particle coating.

According to at least one embodiment of the invention, a magnetic capture device may include a ferromagnetic element and a bioactive coating affixed to the surface of the ferromagnetic element, the ferromagnetic element having a size of at least 2 mm in at least one dimension, and the bioactive coating including a capture element, the capture element itself including at least one of antibodies, aptamers, oligonucleotides, bacteriophages, and molecularly imprinted polymers.

According to further embodiments of the invention, the ferromagnetic element may have a surface area of at least 4 mm², a surface area of at least 1 cm², and/or a size of at least 2 cm in at least one dimension, and/or a shape which is one of rectangular, cylindrical, triangular, and ovoid.

According to a further embodiment of the invention, the ferromagnetic element may include at least one of iron, chromium, aluminum, uranium, platinum copper, cobalt, neodymium, nickel, or magnesium, either in metallic, alloyed, oxide, sulfide, or oxyhydroxide form, and/or a ferrimagnetic/antiferrimagnetic material such as ferrite, magnetite, ulvospinel, hematite, ilmenite, maghemite, jacobsite, pyrrhotite, greigite, troilite, awaruite, wairauite, magnetic steel, chromidur, Silmanal, platinax, bismanol, ultra-mag, vectolite, rectorite, magnadur, lodex, the rare earth elements, goethite, lepidocrocite, or peroxyhyte.

According to a further embodiment of the invention, the bioactive coating may include a functionalizer, the functionalizer being disposed between the capture element and the surface of ferromagnetic element and configured to affix the capture element to the surface of ferromagnetic element.

According to a further embodiment of the invention, the functionalizer may be one of a silicon-based glass, a silane coupling agent, a natural polymer, and a synthetic polymer, such as (3-mercaptopropyl)trimethoxysilane, (3-aminopropyl)triethoxysilane, (3-glycidyloxypropyl)trimethoxysilane, dextran, chitosan, cellulose, polyethylene glycol, polyvinyl alcohol, polylactic acid, polyglycolic acid, alginate, polystyrene, acrylic, polyurethane, polyimide, polyamide, and epoxy.

According to a further embodiment, the magnetic capture device may be utilized with a capture container to capture a target analyte with a mixture. The mixture may be, for example, one of an aqueous liquid, a non-aqueous liquid, an oil-in-water emulsion, a water-in-oil liquid emulsion, an oil, an alcohol, a complex matrix including any of vegetable matter, animal matter, other food or agricultural matter, or a combination thereof. The magnetic capture device may be placed inside the capture container with the mixture and caused to be moved in relation to at least one of the capture container and the mixture. The magnetic capture device may then be recovered with the target analyte affixed thereto.

According to a further embodiment, the target analyte may be later released from the magnetic capture device, and optionally the amount of the target analyte captured may be quantified.

According to another embodiment of the invention, a kit for the capture of biological materials may include a magnetic capture device and a capture container, the magnetic capture device including a ferromagnetic element and a bioactive coating affixed to the surface of the ferromagnetic element and having a size of at least 2 mm in at least one dimension, and the bioactive coating including at least one of the following: antibodies, aptamers, oligonucleotides, bacteriophages, and molecularly imprinted polymers.

According to a further embodiment of the invention, the capture container may be one of a beaker, a bucket, a vial, a test tube, and a cup.

According to a further embodiment of the invention, the capture container and the magnetic capture device may be configured with predetermined geometries such that when said magnetic capture device is utilized in said capture container, an efficient fluid dynamic system is established. An efficient fluid dynamic system may be defined for the purposes of this invention as a system where there is an optimized flow of liquid passing over the magnetic capture device. As described, the magnetic capture device may be agitated, rotated so as to create a vortex in the capture container, or otherwise moved in relation to the capture container, the contents of the container, or both. Such a vortex will have properties dependent on the geometries of the capture container and the magnetic capture device. Thus, pre-determined relative geometries may be chosen to create vortices and fluid dynamic systems as desired by the user.

According to another embodiment of the invention, a kit for the creation of a magnetic capture device may include a magnetic capture device and a functionalizer, the functionalizer being configured to affix a user-chosen capture element to the surface of the magnetic capture device. In such a fashion, a customizable kit may be configured to incorporate any suitable capture element as desired by a user. Such a customizable kit may also include a capture container, and the capture container may be optimized for fluid dynamics as described above.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

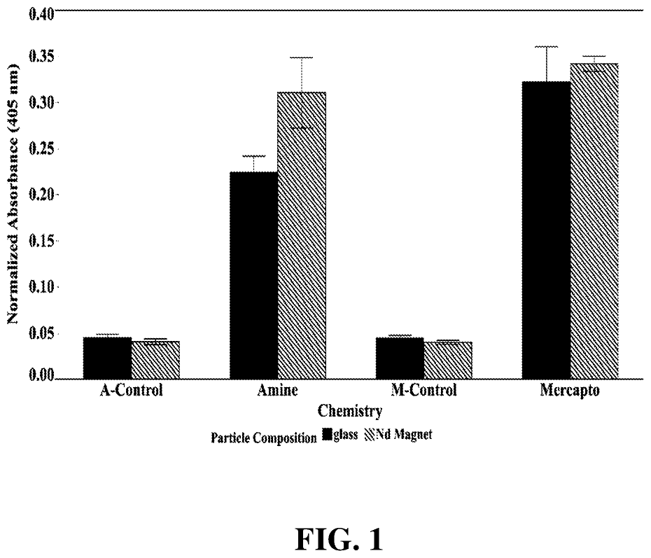

Exemplary FIG. 1 shows a comparison of the attachment of alkaline phosphatase conjugated anti-E. coli antibodies to a glass-encased magnetic stir bar versus a nickel-copper-nickel plated neodymium-iron-boron (NeFeB) magnet when amine and sulfhydryl (mercapto) chemistries are used to functionalize the surfaces. The amine (A) and mercapto (M) controls consisted of cleaned magnets not coated with silane.

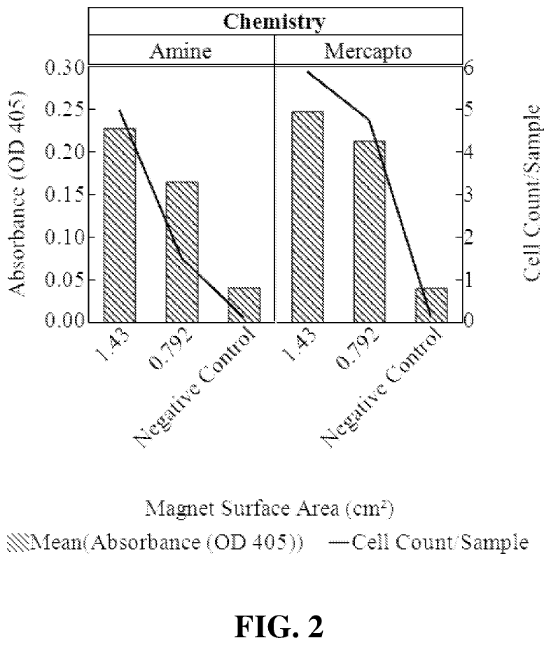

Exemplary FIG. 2 shows the effects of surface functionalization and size on the subsequent capture of Escherichia coli cells by NdFeB magnets. Two different surface areas were tested: 0.792 cm² and 1.43 cm² and their ability to capture E. coli (solid lines) was tested under both amine and mercapto surface functionalizations. The right-hand y-axis relates to the number of cells captured (solid lines) and the left-hand y-axis relates to an absorbance reading which is proportional to the amount of antibody on the surface of the particles. Negative controls consisted of cleaned magnets that did not undergo salinization treatment.

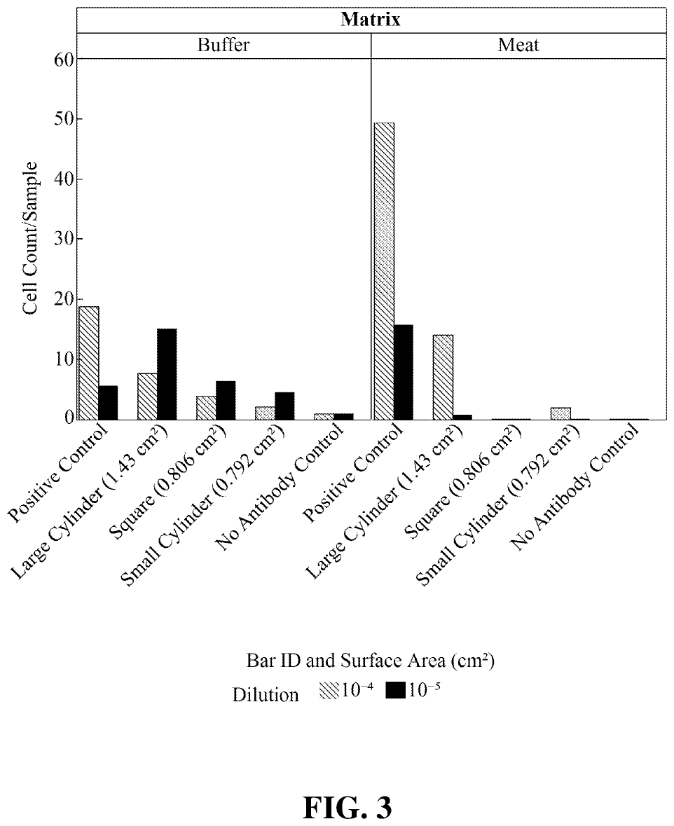

Exemplary FIG. 3 shows a comparison between the number of E. coli cells captured using different sizes and geometries of antibody-coated NdFeB magnets in two different matrixes (buffered peptone water and a ground beef homogenate) and at two different dilutions of the E. coli culture (10⁻⁴ and 10⁻⁵). Positive controls consisted of samples taken directly from the inoculum while negative controls consisted of magnets not coated with antibody.

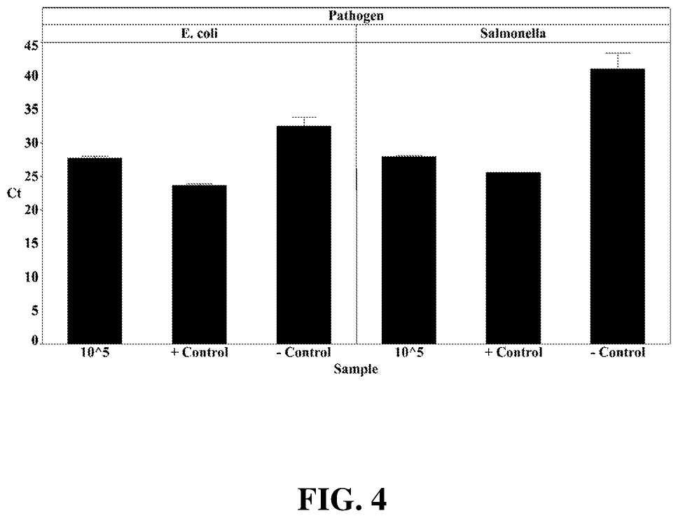

Exemplary FIG. 4 shows a comparison between capture of E. coli and Salmonella enterica analytes, demonstrating the ability of properly-functionalized magnetic capture devices to effectively capture either one.

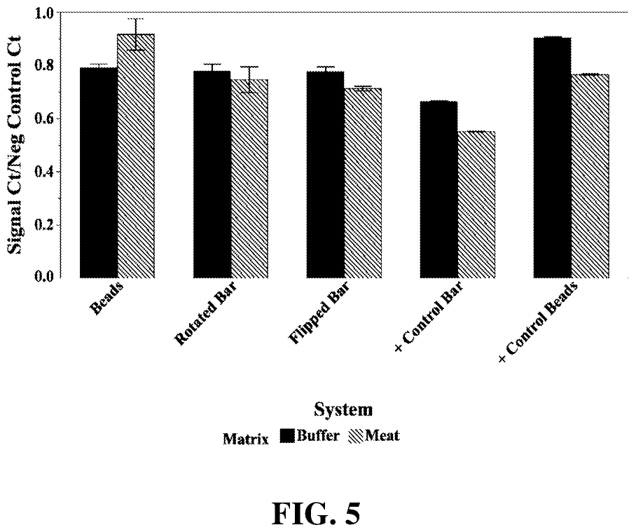

Exemplary FIG. 5 shows a comparison between a magnetic capture device according to the present invention and commercially available magnetic beads in both a buffer and complex (meat) matrix.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Unless defined otherwise, all technical and scientific terms used herein have the same meaning as commonly understood by one of ordinary skill in the art to which the invention belongs. As used herein, the term “about” refers to a quantity, level, value, or amount that varies by as much as 30%, preferably by as much as 20%, and more preferably by as much as 10% to a reference quantity, level, value, or amount. Although any methods and materials similar or equivalent to those described herein can be used in the practice or testing of the present invention, the preferred methods and materials are now described.

The amounts, percentages, and ranges disclosed herein are not meant to be limiting, and increments between the recited amounts, percentages, and ranges are specifically envisioned as part of the invention.

“Optional” or “optionally” means that the subsequently described event or circumstance may or may not occur, and that the description includes instances in which said event or circumstance occurs and instances where it does not. For example, the phrase “optionally comprising X” means that the composition may or may not contain X, and that this description includes compositions that contain and do not contain X.

The term “effective amount” of a compound or property as provided herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein).

The invention illustratively disclosed herein suitably may be practiced in the absence of any element (e.g., method (or process) steps or composition components) which is not specifically disclosed herein.

The term “bioactive” means having an effect on a biological organism or material. An element which is bioactive may, for example, selectively bind or interact with a particular type, species, or genus of biological material or organism.

The term “biological material” means a natural biocompatible material which includes a whole or part of an organism. The term biological material encompasses, for example, microorganisms, cells, tissue, serum, proteins, and nucleic acid constructs.

According to at least one exemplary embodiment, a magnetic capture device may include a ferromagnetic element having a size of at least 2 mm in at least one dimension and a bioactive coating on the surface of the ferromagnetic element. The bioactive coating may include capture or recognition elements, for example antibodies, aptamers, oligonucleotides, bacteriophages, or other suitable capture elements such as molecularly imprinted polymers.

According to another exemplary embodiment of the invention, a kit for capturing biological materials may include a magnetic capture device as described above and a capture container. The capture container and magnetic capture device may be designed with certain predetermined geometries and/or sizes such that when the magnetic capture device is utilized within the capture container, effective capture of one or more biological materials is realized.

According to yet another exemplary embodiment of the invention, a kit for the creation of a magnetic capture device may include a ferromagnetic element and one or more surface functionalizers. Said one or more surface functionalizers may appropriately functionalize the surface of the ferromagnetic element such that a capture element can later be amended onto the functionalized surface using known means by an end user.

The ferromagnetic element may be a solid ferromagnetic magnet. The ferromagnetic element may be made of any suitable ferromagnetic material. In general metals including iron, chromium, aluminum, uranium, platinum copper, cobalt, neodymium, and nickel are magnetic. Compounds and alloys of these materials, including ferrimagnetic and antiferrimagnetic compounds, can also display magnetic properties including, but not limited to ferrites, metallic oxides (such as magnetite, ulvospinel, hematite, ilmenite, maghemite, jacobsite, etc.) metallic sulfides (including pyrrhotite, greigite, troilite, etc.), alloys (including alnico, awaruite, wairauite, magnetic steel, chromidur, silmanal, platinax, bismanol, magnesium-based, vectolite, magnadur, lodex, rare earth, etc.) and magnetic oxyhyrodoxides (including goethitie, lepidocrocite peroxyhyte). Any suitable magnetic material, including those listed above, may be used in the present invention. The ferromagnetic element may have a size of at least 2 mm in at least one dimension and may be of any suitable shape. Exemplary shapes include rectangular, cylindrical, triangular, and ovoid shapes. Other shapes may be used, for example if particular fluidic properties are desired.

The bioactive coating may include a functionalizer and a capture element. The functionalizer may be, for example, a silicon-based glass, a silane coupling agent (such as (3-mercaptopropyl)trimethoxysilane, (3-aminopropyl)triethoxysilane, or 3-(glycidyloxypropyl)trimethoxysilane, etc.), a natural polymer (such as dextran, chitosan, cellulose, etc.) or a synthetic polymer (such as polyethylene glycol, polyvinyl alcohol, polylactic acid, polyglycolic acid, alginate, polystyrene, acrylics, polyurethanes, polyimides, polyamides, epoxies, etc.). The functionalizer may effectively bond one or more capture elements to the surface of the ferromagnetic element. In some embodiments, multiple functionalizers may be used to effectively couple a capture element to the ferromagnetic element.

A capture element according to the present invention refers to a chemical or biological construct that is capable of bonding to or with, or capturing, a target chemical or biological analyte. Examples of capture elements include but are not limited to antibodies, aptamers, oligonucleotides, bacteriophages, and molecularly imprinted polymers.

The capture container may be any suitable container capable of holding the desired amount of liquid to be queried and the magnetic capture device. For example, a capture container may be a beaker, a bucket, a vial, a test tube, a cup, or any other similar container. In some embodiments, a specific capture container may be chosen to be employed in the present invention based on the fluidic properties created by spinning the magnetic capture device in the liquid to be queried in the capture container. For example, to optimize the liquid passing over the magnetic capture device, a particular shape and/or size of capture container may be employed.

In use for capturing a target analyte, the magnetic capture device may be utilized with a capture container to capture a target analyte with a mixture. The mixture may be, for example, one of an aqueous liquid, a non-aqueous liquid, an oil-in-water emulsion, a water-in-oil liquid emulsion, an oil, an alcohol, a complex matrix including any of vegetable matter, animal matter, other food or agricultural matter, or a combination thereof. The magnetic capture device may be placed inside the capture container with the mixture and caused to be moved in relation to at least one of the capture container and the mixture. For example, it may be rotated to create a vortex, caused to be moved by rotating or moving the capture container, or agitated in some other way to enhance the efficiency of capture. The magnetic capture device may then be recovered with the target analyte affixed thereto.

The target analyte may be any suitable chemical or biological entity. Exemplary analytes include, but are not limited to, small and large molecules, including proteins and nucleic acid constructs, cells, and viruses. If desired, the amount of the analyte may be determined by any known or suitable means. It is contemplated that various methods of detection are available for different analyte types and that different methods may be desired depending on the analyte, conditions for testing, time available, and other considerations.

Further embodiments and features of the present invention may be understood from the following examples.

### EXAMPLE 1: SURFACE FUNCTIONALIZATION TRIALS

The effect of two different functionalizers for the magnet surface was investigated. Specifically, both (3-Mercaptopropyl)trimethoxysilane (MPTMS) and (3-Aminopropyl)triethoxysilane (APTES) were used to functionalize a NdFeB magnet. A glass-coated stir bar was also functionalized with the above stated glass coating modifiers in order to benchmark the efficiency of surface modification on the magnet.

Prior to applying the coating chemistry, both the NdFeB magnets and glass coated stir bars were first treated with piranha solution (sulfuric acid and hydrogen peroxide) to remove organic matter and hydroxylate their surfaces. The surfaces were then thoroughly rinsed in water, anhydrous alcohol, and vacuum dried.

To functionalize with APTES, the substrates were completely submerged in a 2% solution of APTES in acetone for 45 seconds. Next, the samples were rinsed well with acetone and dried at 150° C. for 24 hours. Once dried, antibodies were conjugated to the surface using carbodiimide chemistry. A phosphatase-labeled affinity purified antibody to Escherichia coli O157:H7 with an alkaline phosphatase to antibody ratio of 3:1 was activated with 10 molar excess of 1-ethyl-3-(3-dimethylaminopropyl) carbodiimide (EDC) and 25 molar excess N-hydroxysulfosuccinimide (Sulfo-NHS) for 15 minutes, with the excess EDC/NHS subsequently removed using a Zeba Spin Desalting Column, 7K MWCO (ThermoFisher Scientific). The APTES-coated substrate was then submerged in the activated antibody solution for 1 hour and rinsed with phosphate-buffered saline (PBS) containing 0.05% Tween-20. A final rinse was performed immediately before use in PBS.

To functionalize with MPTMS, the substrates were submerged in a 0.1 mM solution of MPTMS in anhydrous ethanol for 30 minutes and air dried. The dried substrate was subsequently immersed in a 1% MPTMS solution in ethanol titrated to a pH of 4.5 using glacial acetic acid for 2 hours. The substrates were then rinsed twice with nanopure water and dried at 150° C. for 24 hours. A phosphatase-labeled affinity purified antibody to E. coli O157:H7 with an alkaline phosphatase to antibody ratio of 3:1 was activated with 20-fold molar excess sulfosuccinimidyl 4-[N-maleimidomethyl]cyclohexane-1carboxylate (Sulfo-SMCC) for 30 minutes and excess SMCC was removed using a Zeba Spin Desalting Column, 7K MWCO (ThermoFisher Scientific). The MPTMS-coated substrate was then submerged in the activated antibody solution as described above for 1 hour and rinsed with a solution of PBS and Tween-20. A final rinse was also performed in PBS immediately before use.

To determine the coating efficiency, a colorimetric assay was conducted. This assay utilized the ability of the alkaline phosphatase that was conjugated to the antibody to produce a color change in order to semi-quantitatively determine the amount of antibody on the magnet. For this assay, each magnet was individually placed into a separate 1.5 mL centrifuge tube. To each tube, 600 μL of a 1 mg/mL p-Nitrophenyl phosphate (pNPP) solution in 0.2 M Tris was added and allowed to incubate for 20 minutes in a dark environment. Following incubation for 20 minutes, the magnets were removed to stop the reaction and the absorbance of 100 μL aliquots which were diluted 10-fold in nanopure water was measured at 405 nm using a Tecan Safire 2. As controls, two of each type of substrate were cleaned but not coated with silane. The results are shown in FIG. 1. As can be seen, the MPTMS-coated NdFeB magnets performed the best, but all tested substrate-functionalizer combinations tested performed significantly better than the non-coated controls.

### EXAMPLE 2: CAPTURE OF TARGET BACTERIUM

The effects that the different functionalized surfaces had on the ability of the NdFeB magnets to capture E. coli cells was also tested. NdFeB magnets coated using both APTES and MPTMS chemistry and antibodies were tested.

Overnight cultures of Escherichia coli O157:H7-PC cells were diluted to working concentrations (ca. 10,000-100,000 CFU/mL in 0.1% buffered peptone water). A 10 mL aliquot of the cell solution was dispensed into each well of a 6-well culture plate and the individual antibody-coated NdFeB magnets were added to the plates. The magnets were stirred at 150 rpm for 10 min. A wash step consisting of stirring the bar in 10 mL of phosphate buffered saline (PBS, pH 7) for 2 min was performed to remove any unbound material that was carried over. The magnets were then removed from the wash solution and placed into a 0.2 mL tube using another magnet to facilitate the particle's transfer. 150 μL of PCR-grade water was added to the tube. Tubes were boiled at 100° C. for 10 minutes and then cooled to 4° C. in a BioRad T100 Thermal cycler in order to lyse the captured cells. The magnets were subsequently removed and the tubes were spun at 10,000×g for 5 min to remove cell debris with the supernatant being placed into a clean microfuge tube. From there, 8 μL of the supernatant was placed into a MicroAmp Fast Reaction tube (Applied Biosystems) along with 10 μL of Dynamo Flash master mix (ThermoFisher), 0.5 μL of STEC-Shuffle-F primer, 0.5 μL of STEC-Shuffle-R primer, 0.25 μL of STEC-Shuffle-P (probe), 0.4 μL of 50×ROX and 0.35 μL of distilled H₂O. This primer/probe combination amplifies a genomic marker sequence that was placed into the E. coli O157:H7-PC strain utilized in these experiments and allows it to be differentiated from E. coli O157:H7 strains that may be present naturally.

Quantitative PCR (qPCR) was performed, and antibody deposition was also determined for these magnets using the colorimetric assays described above. The results, shown in FIG. 2, demonstrate that usage of the MPTMS chemistry was superior for the functionalization of the NdFeB magnets in terms of both antibodies deposited and cells captured and thus was used in subsequent experiments.

### EXAMPLE 3: CAPTURE OF TARGET BACTERIUM IN COMPLEX MATRICES

The ability of the NdFeB magnets to capture E. coli O157:H7 in both 0.1% buffered peptone water and a complex food matrix was also tested, as well as the effect of the geometry of the substrate.

For the buffered peptone water assays, overnight cultures of Escherichia coli O157:H7-PC cells were diluted to working concentrations (ca. 10,000-100,000 CFU/mL in 0.1% peptone buffer water). A 35 mL aliquot of the cell solution was dispensed into multiple petri plates and the individual MPTMS antibody-coated NdFeB magnets were added to the plates at room temperature. The magnets were stirred at 350 rpm for 10 min and then washed with 35 mL of PBS to remove any unbound material that was carried over. The magnets were then removed from the wash solution and placed into a 0.2 mL tubes using another magnet to facilitate the transfer. 120 μL of PCR-grade water was added to the tubes. Cells were lysed by boiling and qPCR was performed using the supernatant as described above.

The complex food matrix was tested in an identical manner as that of the buffered peptone water assays except the complex food matrix consisted of 35 mL aliquots of the following: a solution was made containing an overnight culture of Escherichia coli O157:H7-PC cells diluted to working concentrations (ca. 10,000-100,000 cfu/mL) in 250 mL of 0.1% buffered peptone water that was processed in a Stomacher bag with 81 g of ground beef for 2 min at normal speed. Positive controls consisted of samples taken directly from the diluted overnight culture of Escherichia coli O157:H7-PC cells while negative controls consisted of magnets not coated with antibodies.

The results from this assay, shown in FIG. 3, demonstrate the ability of the magnet to capture E. coli O157:H7 in both buffer ground meat matrices, the capacity of the magnet to concentrate the cells (comparing the 10⁻⁵ dilution in buffer on the large cylinder and the positive control), and that different sizes/geometries of bars have different capture efficiencies, at least for the present analyte and container.

### EXAMPLE 4: CAPTURE OF MULTIPLE PATHOGENS

The ability of the magnetic capture device to capture different pathogens was investigated by testing for two different pathogens: E. coli and Salmonella enterica. NdFeB magnets coated with silane were conjugated with either anti-E. coli or anti-Salmonella antibodies before being subjected to a solution of either E. coli (˜1×10⁵ CFU/mL) or S. enterica (˜2×10⁵ CFU/mL). Negative controls (−Control) consisted of MPTMS coated NdFeB magnets exposed to ˜1-2×10⁵ CFU/mL of pathogen that were silane coated but were not subjected to antibody conjugation. Positive controls (+Control) consist of an aliquot of each pathogen dilution to which the magnets were subjected.

Cycle threshold (Ct) values representing the number of cells captured by the antibody-coated NdFeB magnets were measured via qPCR with primers/probes specific to the cells of interest, with the results shown in FIG. 4. As can be seen, average Ct values dropped in the test samples as compared to the negative control, demonstrating capture of the analytes on those test samples.

### EXAMPLE 5: COMPARISON AGAINST BEADS

Currently, commercially-available superparamagnetic particles or beads are used for separation. The present invention was compared against commercial beads in both 30 mL of a buffer and 30 mL of a complex matrix (ground beef homogenate, prepared as described above). To ensure as close of a comparison as possible, 6 μL of the prepared beads (each one 4.5 μm-diameter) was used so that the surface areas of both the beads used and the bar were equivalent. In addition, both the prepared commercial beads and the magnetic capture device according to the present invention were exposed to the matrix for 10 min.

The preparation and capture procedure for the magnetic capture device according to the present invention was identical to that described above.

To prepare the magnetic beads, tosylactivated M-280 Dynabeads (Invitrogen,) were conjugated with BacTrace Anti-Escherichia coli O157:H7 Antibody (SeraCare, Milford Mass.) using the guidelines provided in the product technical data sheet. Control beads were prepared in the same fashion except the antibody was substituted with a “surrogate” protein, bovine serum albumin.

The capture procedure for the beads follows. Because the meat matrix contained a lot of particulate matter, care was taken to avoid any carry over of this material into the final analyzed sample; thus, the beads were processed through a MACS Large Cell Separation Column (Miltenyi Biotech) whereas magnetic bars were simply removed from the matrix and placed into a clean container using another magnet to facilitate their movement. Processing through the MACS columns consisted of securing the column using a magnetic holder and passing 10 mL of 0.1 M sodium phosphate buffer, pH 7.4, followed by 2 mL of 0.5 wt % bovine serum albumin in PBS, pH 7.4 across the beads. Beds were eluted from the column by removing the column from the magnetic holder and placing 150 μL nuclease-free water into the column and flushing out the beads in a 0.2 mL tube using the supplied plunger. The beads were then processed in a fashion similar to the magnetic bars described as follows: captured cells were lysed via the boil method and then cooled. The tubes were spun at 10,000×g for 5 min to pellet cell debris and then placed against a magnet to facilitate removal of the supernatant without contamination from the beads with the supernatant being placed into a clean microfuge tube. From there, 8 μL of the supernatant was used along with 10 μL of Dynamo Flash master mix (ThermoFisher), 0.5 μL of STEC-Shuffle-F primer, 0.5 μL of STEC-Shuffle-R primer, 0.25 μL of STEC-Shuffle-P (probe), 0.4 μL of 50×ROX and 0.35 μL of dH₂O for gene amplification. This primer/probe combination amplifies a genomic marker sequence that was placed into the E. coli O157:H7-PC strain utilized in these experiments and allows it to be differentiated from E. coli O157:H7 strains that may be present naturally. This was particularly important for the work performed with meat samples.

For the magnetic capture device studied, three different variations of movement were tested: allowing the magnetic bar to remain stationary while capturing; spinning (rotating) the magnetic bar using a magnetic stir plate while capturing; and flipping (turning the tubes in which the magnetic bar had been placed end-over-end, thereby externally agitating the capture container and the magnetic capture device therein) the bar while capturing.

The results of this study are shown in FIG. 5. As can be seen, the magnetic capture device performed approximately as well as the magnetic beads tested. However, this performance was achieved with a significantly simpler capture procedure (simply removing the magnetic capture device and rinsing, as opposed to the procedure outlined above for the beads).

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1: A magnetic capture device, comprising:
a ferromagnetic element; and
a bioactive coating affixed to the surface of the ferromagnetic element,
wherein the ferromagnetic element has a size of at least 2 mm in at least one dimension, and
wherein the bioactive coating comprises a capture element, the capture element comprising at least one of: antibodies, aptamers, oligonucleotides, bacteriophages, and molecularly imprinted polymers.

2: The magnetic capture device of claim 1, wherein the ferromagnetic element has a surface area of at least 4 mm².

3: The magnetic capture device of claim 1, wherein the ferromagnetic element has a surface area of at least 1 cm².

4: The magnetic capture device of claim 1, wherein the ferromagnetic element has a size of at least 2 cm in at least one dimension.

5: The magnetic capture device of claim 1, wherein the magnetic capture device has a shape which is one of rectangular, cylindrical, triangular, and ovoid.

6: The magnetic capture device of claim 1, wherein the ferromagnetic element comprises at least one of iron, chromium, aluminum, uranium, platinum copper, cobalt, neodymium, nickel, magnesium, ferrite, magnetite, alnico, ulvospinel, hematite, ilmenite, maghemite, jacobsite, pyrrhotite, greigite, troilite, awaruite, wairauite, magnetic steel, chromidur, silmanal, platinax, bismanol, ultra-mag, vectolite, magnadur, lodex, the rare earth elements, goethitie, lepidocrocite, and peroxyhyte.

7: The magnetic capture device of claim 1,
wherein the bioactive coating further comprises a functionalizer, and
wherein the functionalizer is disposed between the capture element and the surface of ferromagnetic element, and the functionalizer is configured to affix the capture element to the surface of ferromagnetic element.

8: The magnetic capture device of claim 7, wherein the functionalizer is one of a silicon-based glass, a silane coupling agent, a natural polymer, and a synthetic polymer.

9: The magnetic capture device of claim 8, wherein the functionalizer is one of (3-mercaptopropyl)trimethoxysilane, (3-aminopropyl)triethoxysilane, (3-glycidyloxypropyl)trimethoxysilane, dextran, chitosan, cellulose, polyethylene glycol, polyvinyl alcohol, polylactic acid, polyglycolic acid, alginate, polystyrene, acrylic, polyurethane, polyimide, polyamide, and epoxy.

10: A method of capturing a target analyte, the method comprising:
providing a capture container;
placing inside the capture container the magnetic capture device of claim 1 and a mixture, the mixture containing the target analyte;
causing the magnetic capture device to be moved in relation to at least one of the capture container and the mixture; and
recovering the magnetic capture device,
wherein the capture element of the magnetic capture device is effective to capture the target analyte.

11: The method of claim 10, further comprising:
releasing the target analyte from the magnetic capture device.

12: The method of claim 10, further comprising:
quantifying the amount of target analyte captured.

13: A kit for the capture of biological materials, comprising:
a magnetic capture device; and
a capture container,
wherein the magnetic capture device comprises a ferromagnetic element and a bioactive coating affixed to the surface of the ferromagnetic element,
wherein the ferromagnetic element has a size of at least 2 mm in at least one dimension, and
wherein the bioactive coating comprises at least one of: antibodies, aptamers, oligonucleotides, bacteriophages, and molecularly imprinted polymers.

14: The kit for the capture of biological materials of claim 13, wherein the capture container is one of a beaker, a bucket, a vial, a test tube, and a cup.

15: The kit for the capture of biological materials of claim 13, wherein the capture container and the magnetic capture device are configured with predetermined geometries such that when said magnetic capture device is utilized in said capture container, an efficient fluid dynamic system is established.

16: A kit for the creation of a magnetic capture device, comprising:
a magnetic capture device; and
a functionalizer,
wherein the ferromagnetic element has a size of at least 2 mm in at least one dimension,
wherein the functionalizer is configured to affix a user-chosen capture element to the surface of the magnetic capture device.

17: The kit of claim 16, further comprising:
a capture container.

18: The kit of claim 17, wherein the capture container and the magnetic capture device are configured with predetermined geometries such that when said magnetic capture device is utilized in said capture container, an efficient fluid dynamic system is established.
